(19) United States
(12) Patent Application Publication
(10) Pub. No.: US 2005/0079566 A1
(43) Pub. Date: Apr. 14, 2005

(54) Analytical methods for determination of proteolytic cleavage at specific sites

(76) Inventor: Nigel Robert Caterer, Copenhagen (DK)

Correspondence Address: Licata & Tyrrell P.C., 66 East Main Street, Marlton, NJ, 08053, US

(21) Appl. No.: 10/938,019

(22) Filed: Sep. 10, 2004

Related U.S. Application Data
(60) Provisional application No. 60/501,882, filed on Sep. 10, 2003.

(51) Int. Cl. G01N 33/53; G01N 33/537; G01N 33/543

(52) U.S. Cl. 435/7.92

(57) ABSTRACT

Methods are provided for determining activity or concentration of an analyte in a sample based upon site-specific enzymatic cleavage of an intact substrate by the analyte or by an enzyme activated by the presence of the analyte.

### INTRODUCTION

This patent application claims the benefit of U.S. Provisional Application Ser. No. 60/501,882, filed Sep. 10, 2003, the teachings of which are incorporated herein in their entirety.

### FIELD OF THE INVENTION

The present invention provides analytical methods of high sensitivity for determining site-specific proteolytic cleavage of a substrate by an enzyme and hence the activity of steps in proteolytic enzyme cascades or activation systems. These methods are thus useful as a means of measuring the concentration of analytes that trigger and/or are involved in such systems. The analytical methods of the present invention are particularly useful in the fields of biochemistry and medicine. In these methods, monoclonal antibodies and/or other specific binding molecules are used to determine proteolytic cleavage at specific sites of peptide or protein substrates. This permits the sensitive detection and quantification of analytes which trigger and/or are involved in proteolytic enzyme cascade amplification systems including, but not limited to, the blood clotting system, the horseshoe-crab amebocyte lysate clotting system, fibrinolysis, and the complement system.

### BACKGROUND OF THE INVENTION

Synthetic chromogenic peptide sequences have been used for detecting site-specific enzymatic cleavage. In these methods, the amino-acid residue immediately downstream of the cleavage site in the synthetic peptide is replaced by a chemical moiety, which, upon hydrolysis, undergoes a detectable change in its light absorption characteristics so as to generate a color.

One disadvantage of the use of such chromogenic peptide substrates is that the amino-acid residue(s) immediately downstream of the cleavage site cannot be used to increase the specificity of the substrate, because of their replacement with the chromogenic moiety. Another disadvantage of these methods is that there are limited numbers of appropriate chromogenic moieties available. A further disadvantage is that each substrate molecule that is split generates only one molecule producing the color. This makes such methods relatively insensitive, and a prolonged reaction time may be needed to obtain a reliably measurable signal.

Specific monoclonal antibodies against neo-epitopes formed by the specific enzymatic cleavage of a protein substrate have also been used. In these methods, monoclonal antibodies are selected empirically on the basis that they react with the product much more strongly than with the substrate. The precise nature of the neo-epitope with which they react, however, is in many cases not known. In some cases the neo-epitopes are believed to be structures resulting from conformational changes due to the site-specific cleavage. In other cases, it is known that the antibody reacts with a newly exposed C- or N-terminus of the proteolytically cleaved substrate, for example, the newly exposed C-terminal octapeptide of C3a des-Arg, generated by the action of C3 convertase and carboxypeptidase N on C3 of the complement system (Zilow G, Naser W, Rutz R, Burger R, 1989, Quantitation of the anaphylatoxin C3a in the presence of C3 by a novel sandwich ELISA using monoclonal antibody to a C3a neoepitope. Journal of Immunological Methods 121:261-268, 1989; and EP0312645: Antigenic peptides from complement factor C3a, use of peptides, cell lines, antibodies against the peptides and utilisation of the antibodies. Inventors: Burger R, Naser W, 1989). Quantification of site-specific proteolytic activity by a binding molecule known in prior art to bind to a newly exposed terminus of a native substrate is specifically excluded from the present invention, when the native substrate is not bound to a solid phase according to the present invention.

In the present invention, detection of site-specific proteolytic cleavage is performed by means of a binding molecule which is specific to the cleaved substrate. This permits the use of a considerably larger number of substrate molecules and detection systems, including detection systems with enzyme-linked or other means of immunochemical amplification, in which many reporter molecules are produced per molecule of cleaved substrate, allowing for a much higher sensitivity of detection and quantification of the specific protease activity.

In the present invention, binding molecules, including monoclonal antibodies, are used which are specific for new terminal sequences exposed upon proteolytic cleavage of an intact peptide or protein substrate and which do not react with the same sequences when they form an integral part of the protein chain in the uncleaved intact substrate. An aspect of the present invention is that the intact substrate is attached to a solid phase so that the cleaved substrate, the binding molecule that specifically attaches to the cleaved substrate, and any signal amplification molecules are also associated with the solid phase.

### SUMMARY OF THE INVENTION

The present invention relates to analytical methods for determining site-specific enzymatic cleavage of protein or peptide substrates by generating and/or selecting binding molecules specific for the free ends of newly exposed terminal sequences of the substrates following proteolytic cleavage. Binding molecules for use in the present invention do not react with the uncleaved intact substrate or with terminal sequences extended by additional chemical moieties that can be split off by the site-specific enzyme. The binding molecules can be linked to a variety of amplification systems providing sensitive detection of the site-specific cleavage.

Accordingly, an aspect of the present invention relates to a method for determining activity or concentration of an analyte in a sample based upon site-specific enzymatic cleavage of a protein or peptide substrate, hereinafter referred to as intact substrate, either by the analyte itself, if this is a site-specific proteolytic enzyme, or by a site-specific proteolytic enzyme which is activated in consequence of the presence of the analyte. In this method, a binding molecule specific for a free end of a newly exposed terminal sequence of the intact substrate following proteolytic cleavage of the intact substrate, referred to hereinafter as the cleaved substrate, by the site-specific enzyme is first generated and/or selected. A sample suspected of containing the site-specific proteolytic analyte or an analyte mixed with the appropriate proenzyme system in which it triggers the generation of the site-specific proteolytic enzyme, is then contacted with the intact substrate under conditions which promote or induce site-specific proteolytic cleavage of the intact substrate by the site-specific proteolytic enzyme, thereby producing the cleaved substrate. The intact substrates used in the present invention are attached to a solid phase before the addition of sample and are not derived from the sample. Any site-specific proteolytically cleaved substrate is then contacted with the binding molecule and any binding of the binding molecule to the site-specific proteolytically cleaved substrate is measured to determine activity or concentration of the analyte in the sample. In a preferred embodiment, the binding molecule is linked to an amplification system to increase sensitivity.

### BRIEF DESCRIPTION OF THE FIGURES

FIG. 1 is a line graph showing results from an enzyme-linked immunosorbent assay (ELISA) comparing the binding of the monoclonal antibody HYB 100-01 applied in serial doubling dilutions of culture supernatant (CS) to wells coated at 1 μg/ml with either a C-terminal (Peptide SEQ ID NO:11, shown as a solid line with a cross) or N-terminal (Peptide SEQ ID NO:10, shown as a dotted line with a filled square) synthetic peptide from the sequence of peptide c from the coagulogen of Limulus polyphemus amebocyte lysate (LAL).

FIG. 2 is line graph showing results from an ELISA comparing the binding of monoclonal antibody HYB 100-01 applied in serial doubling dilutions of CS to wells coated with a peptide (Peptide SEQ ID NO:11, shown as solid lines) from the C-terminal region of peptide c derived from LAL coagulogen or coated with a peptide of the same amino-acid sequence extended by two amino-acid residues (Gly-Phe) at the C-terminus (Peptide SEQ ID NO:12, shown as dotted lines). The peptides were coated onto the ELISA wells at 20 μg/ml (shown as a cross), 5/ml (shown as an open square) and 0.8 μg/ml (shown as a solid circle).

FIG. 3 is a line graph showing results from an ELISA comparing the binding of purified monoclonal antibody HYB 100-01 in non-conjugated (HYB 100-01, shown as a dotted line with a solid diamond) and peroxidase-conjugated (HYB 100-01-POD, shown as a solid line with a cross) forms, added in serial doubling dilutions to streptavidin-coated wells to which peptide SEQ ID NO:11 had been applied at a concentration of 100 ng/ml.

FIG. 4 is a line graph showing results from an ELISA showing the dose-response curves for Control Standard Endotoxin obtained with different dilutions of Tachypleus tridentatus amebocyte lysate (TAL) reagent. The TAL reagent is diluted 1 part in 12 (shown as a cross), 15 (shown as a solid circle), 30 (shown as an open square) and 40 (shown as an open triangle). The substrate peptide (SEQ ID NO:12) is bound to a streptavidin Immobilizer plate at a concentration of 40 ng/ml.

FIG. 5 is a line graph showing results from an ELISA showing the ability of peroxidase-conjugated monoclonal antibody HYB 100-01-POD to bind peptide SEQ ID NO:11 coated directly to an Immobilizer plate (Nunc) at a concentration of 31.25 μg/ml (Shown as a solid line with a solid diamond). The binding of HYB 100-01-POD to an uncoated surface (no peptide) is also shown as a dashed line with a cross.

FIG. 6 is a line graph showing results from an ELISA showing the binding of antibody HYB 100-01-POD applied at 0.5 μg/ml to wells coated with different peptides at different concentrations and treated with activated TAL. The coated peptides were peptide SEQ ID NO:11 (shown as a solid diamond), peptide SEQ ID NO:13 (shown as an open circle), peptide SEQ ID NO:14 (shown as a solid square), peptide SEQ ID NO:15 (shown as an open triangle) and peptide SEQ ID NO:16 (shown as a cross).

FIGS. 7a and 7b are line graphs showing results from an ELISA showing the dose-response curve for Control Standard Endotoxin (CSE) reacted with TAL reagent (diluted 1 in 12) in wells of an Immobilizer plate (Nunc) coated directly with the substrate peptide (SEQ ID NO:13) at a concentration of 0.3 μg/ml. FIG. 7a shows the response curve to a CSE concentration of 1.0 EU/ml. FIG. 7b shows the same curve, focusing on the response up to 0.1 EU/ml.

### DETAILED DESCRIPTION OF THE INVENTION

Many proteins that participate in enzymatic activation cascades or systems are proenzymes that are activated by site-specific proteolytic cleavage by the enzyme generated in the preceding step of the cascade. The activated enzyme thus produced is then able to act on the proenzyme substrate of the next step of the cascade, each step producing an amplification over the preceding step because each enzyme molecule produced activates many molecules of proenzyme of the next step. The final step is to generate a large number of effector molecules that implement the physiologic or pathophysiologic functions of the cascade.

The present invention provides analytical methods of high sensitivity for determining the activity and/or concentration of analytes in these proteolytic enzyme cascades or systems based upon measurement of proteolytic cleavage at specific sites of protein or peptide substrates, hereinafter referred to intact substrates. In most cases the complete amino-acid sequence of an intact substrate is known, as is the cleavage site and the sequences of the newly generated N- and C-termini of cleaved substrate on either side of the site. Thus, it is possible to synthesize peptides corresponding to these termini and the therefore the cleaved substrate and to generate and/or select molecules which specifically bind to the cleaved substrate.

Accordingly, in the methods of the present invention, binding molecules are generated and/or selected that bind specifically to peptides corresponding to the newly generated N- or C-termini of a cleaved substrate. These peptides will typically be synthesized chemically, but the method by which peptides of the appropriate structure are obtained is not material to the invention.

Thus, the term “synthetic” when applied to a peptide substrate herein includes the term “recombinant” in the sense of being produced by recombinant DNA technology, as the precise method by which the required substrate is produced is not material to the invention. The term “protein”, when applied to a substrate herein includes both an entire protein and any protein fragment that is larger than one that may conveniently be synthesized chemically, and such proteins or protein fragments may be prepared from either native or recombinant material.

General examples of synthetic intact substrates useful in the present invention are depicted below in Formula (a) and Formula (b)
X1-X2-X3-X4-X5-X6-X7-X8-X9 (a)
X1-X2-X3-X4-hapten (b)
In these formulas, the amino-acid residues primarily responsible for defining the site of specific proteolytic cleavage are shown in italics, and the blocking group that prevents union of the binding molecule with the uncleaved, intact substrate is underlined.

When the binding molecule used to detect site-specific proteolysis is specific for the newly exposed C-terminus of the synthetic substrate, use of an intact substrate of Formula (a) is preferred. The intact substrate thus comprises, for example, amino acid residues, X1-X8, the last of which is amide-linked to a blocking group, X9. X1-X4 can be chosen to match the corresponding residues of the native substrate, but they can also be non-matching residues chosen for favorable properties of cleavage efficiency and specificity, stability, solubility and antigenicity. X5-X8, in italics, are the 4 amino-acid residues that are most responsible for defining the site of proteolytic cleavage, which takes place between X8 and X9, and can be chosen to match the corresponding residues of the native substrate, but they can also be non-matching residues chosen for favorable properties of cleavage efficiency and specificity, stability, solubility and antigenicity. The binding molecule is generated and selected to bind specifically to X1-X8, and not to X1-X9. X9, underlined, is the blocking group which is removed by site-specific proteolysis between X8 and X9. X9 can be one or more amino-acid residues, but can also be another chemical moiety linked to X8 by an amide bond that is cleaved by site-specific proteolysis. The presence of X9 prevents the binding molecule from binding to the intact substrate and its removal enables the binding molecule to bind to cleaved substrate X1-X8.

When the binding molecule used to detect site-specific proteolysis is specific for the newly exposed N-terminus of the intact substrate, use of a substrate of Formula (b) is preferred. Intact substrates of Formula (b) may comprise as little as 3 or 4 amino-acid residues, in this case X1-X4, that define the site of proteolytic cleavage, linked via an amide bond at its C-terminus to the primary amino group that constitutes the N-terminus of a hapten. The hapten can be a peptide sequence chosen to match the initial, preferably at least 3, or more preferably at least 7, amino-acid residues of the newly exposed N-terminus of the native substrate, but the hapten can also consist of non-matching amino-acid residues including non-naturally occurring amino-acid residues, or it can be any chemical compound that can be linked to the C-terminus of the site-defining amino-acid residues by means of a bond that is cleaved by the site-specific proteolytic enzyme. The hapten, whatever its nature, is chosen for favorable properties of stability, solubility and its capacity to generate antibodies of high affinity and specificity against itself, including antibodies that will not bind to the hapten when it is linked to the C-terminus of the site-defining amino acid sequence. X1-X4, in italics and underlined, is hence at one and the same time the 4 amino-acid residues that define the site of proteolytic cleavage, between X4 and the hapten, and the blocking group that prevents the binding molecule from binding to the intact substrate. The binding molecule is generated and selected to bind specifically to the hapten, and not to the intact substrate. X1-X4 is removed by site-specific proteolysis, thus exposing the hapten and making it available for binding by the binding molecule.

Binding molecules selected for use in the present invention react only with free end of the terminus and not with the terminus extended by one or more amino-acid residues or by another chemical group, nor with the intact macromolecular substrate. To achieve a high affinity and specificity for the terminal amino acid sequence, the peptide against which the binding molecule is generated and selected typically comprises at least the 4 terminal amino-acid residues, more preferably at least the 8 terminal amino-acid residues in relation to the cleavage site of the intact substrate.

The binding molecules used in the present invention preferably comprise monoclonal antibodies or fragments thereof that include the binding site, antibodies or fragments thereof selected by phage display, yeast display or other selection systems, or other binding molecules that can be specifically selected for this purpose. Because the specific cleavage sites of the intact substrates are usually defined by the amino-acid sequence on the N-terminal side of the site, the most straightforward procedure for generating binding molecules useful in the methods of the present invention is to raise antibodies against the newly generated C-terminus of the cleaved substrate which ends in that sequence. However, it is also possible to generate antibodies against the newly generated N-terminus of the cleaved substrate.

In one embodiment of the present invention, an intact substrate is used to determine the activity or concentration of an analyte in a sample. When the binding molecule is generated and/or selected to be specific against the newly generated C-terminus which ends at the cleavage site, an intact substrate can be used which terminates in the same amino-acid sequence on the N-terminal side of the cleavage site, with the addition of a C-terminally attached extending or blocking group that is split off by the proteolytic activity to be measured. This extending group is referred to as a blocking group because its presence blocks the binding of the binding molecule to the intact substrate (see Formula (a)). When the binding molecule is generated and/or selected to be specific against the newly generated N-terminus, the blocking group at the N-terminus of an intact substrate must include the whole site-defining amino-acid sequence, often amounting to 3 or 4 amino-acid residues (see Formula (b)).

In many cases a wide variety of amino-acid sequences can be present on the opposite side of the cleavage site from the site-defining amino-acid sequence without interfering with the efficiency of cleavage. For example, an artificial amino-acid sequence can be chosen for use in the intact substrates which is unlikely to occur in the biologic components of the system to be analyzed. When a newly generated N-terminus is to be detected, this sequence can include, next to the cleavage site or in the immediately following positions, a non-naturally occurring chemical group chosen to facilitate the production of molecules that bind with very high specificity and affinity to the artificial sequence with its special chemical group, but not to the same structure when extended by the site-defining amino-acid sequence. Indeed, the hapten linked to the C-terminus of the site-defining amino acid sequence need not be a peptide at all, but can be any chemical compound that can be linked to the C-terminus of the site-defining amino acid residues by means of a bond that is cleaved by the site-specific proteolytic enzyme. The hapten, whatever its nature, can be chosen for favorable properties of cleavage efficiency and specificity, stability, solubility and its capacity to generate binding molecules of high affinity and specificity against itself, including binding molecules that will not bind to the hapten when it is linked to the C-terminus of the site-defining amino acid sequence. This means that the same highly selected binding molecule can be used to analyze cleavage at a wide variety of different specific sites, using as intact substrate the hapten, whether or not peptide in nature, with its characteristic chemical group preceded by the specific site-defining amino-acid sequence in question. Such intact hapten substrates with different site-defining amino-acid sequences can be synthesized at low cost.

An intact substrate for this use is preferably attached to a solid phase via the terminus opposite to the extending sequence or blocking group, so that the specific cleavage site is exposed to enzymatic attack. A spacer molecule may intervene between the intact substrate and the solid phase to ensure adequate, flexible exposure to the active site of the enzyme. There are many ways of achieving this, well known to those skilled in the art; one simple method is to couple biotin or biocytin to the terminus of the intact substrate that lies opposite to the extending sequence or blocking group and to bind the biotin moiety to a streptavidin-coated solid phase.

In another embodiment, the methods of the present invention involve use of an intact protein substrate or a large fragment thereof that includes the specific cleavage site. The protein or fragment thereof is attached to the solid phase by direct adsorption or via streptavidin and biotin or biocytin, or in any manner that leaves a sufficient number of specific cleavage sites exposed to the proteolytic activity to be measured. Generation of the new N- or C-termini then allows reaction with the appropriate specific binding molecules, provided that these termini are adequately exposed in the new structure of the attached product.

When the reaction mixture contains a significant concentration of the natural endogenous substrate of the enzymatic reaction, sensitivity of the method may be limited by substrate competition. Sensitivity of such assays may be increased, however, by increasing the availability of solid-phase-bound intact substrate. Further, in some assays the endogenous substrate can be removed by prior treatment, or blocked by the addition of an excess of antibody that binds to the endogenous substrate, but not to the synthetic intact substrate used in the present invention, in such a manner as to prevent access of the enzyme to the specific cleavage site.

Binding of the specific binding molecules to the newly generated termini can be detected and quantified by various means, well known to those skilled in the art, which amplify detectable binding. These include, but are in no way limited to, direct labeling of the binding molecule with a detectable tracer, which may be biotin-related, radioactive, fluorescent, or enzymatic; the use of a similarly labeled antibody against the molecule; and the use of various enzymatic amplification systems including avidin/biotinylated enzyme complexes.

Another method of detecting specific binding, which forms part of the preferred embodiment of this invention, is by coating the intact substrate and binding molecule onto different populations of microparticles or microspheres in suspension. Binding of the binding molecule to the cleaved substrate is then revealed by aggregation of particles that can be detected by light scattering (nephelometry or turbidimetry). This has the advantage of being readily adapted to rapid, automated analytical procedures carried out at the point of care of human patients.

Amplification techniques greatly increase the sensitivity of the methods of the present invention as compared to prior art methods. The increased sensitivity also results in a considerable reduction in the consumption of expensive biological reagents such as horseshoe-crab amebocyte lysate, reducing the cost of analysis and the hazard to the survival of the endangered species presented by the collection of this reagent.

The following nonlimiting exemplary embodiment is provided to further illustrate the present invention. As will be understood by those of skill in the art upon reading this disclosure, the detailed methodologies disclosed below can be routinely adapted in accordance with teachings herein, without any undue experimentation, to other uses including, but in no way limited to, measurement of site-specific protease activities in samples of bodily fluids.

### EXAMPLE

A Horseshoe-Crab Amebocyte Lysate System Used to Detect and Quantify Bacterial Endotoxin

A preferred embodiment of the present invention is described in relation to the use of horseshoe-crab amebocyte lysate as an amplifying proteolytic enzyme cascade system to detect and quantify bacterial endotoxin in aqueous samples.

The amplifying enzyme cascade in horseshoe-crab amebocyte lysate is triggered by the binding of endotoxin to factor C, which then activates a succession of proteolytic proenzyme steps resulting in the generation of large amounts of the final proteolytic enzyme of the cascade, known as “clotting enzyme”. This cleaves the clot-forming substrate, coagulogen, at two specific sites in the N-terminal region of the molecule to liberate a 28-amino-acid peptide, peptide C. The remaining fragments of coagulogen remain united by a disulfide bridge and adopt a new conformation in which successive molecules, now called coagulin, unite to form a clot. A current analytical method employing this system to detect endotoxin uses a synthetic chromogenic peptide substrate analogous to the C-terminus of peptide C from the Atlantic horseshoe crab, Limulus polyphemus, bearing a chromogenic moiety at its C-terminus. The formation of active clotting enzyme is detected by the hydrolysis of this moiety to generate a color.

In relation to its endogenous coagulogen substrate, Limulus clotting enzyme is specific for the two cleavage sites delimiting peptide C. These sites are defined by a sequence of four amino acid residues N-terminal to the cleavage site, the last and most essential of these being arginine. However, the specificity of Limulus clotting enzyme for a determined sequence of four amino acid residues is not absolute. It acts equally well on coagulogens from the Pacific or South China Sea horseshoe crabs Tachypleus tridentatus, T. gigas and Carcinoscorpius rotundicauda, in which the fourth position N-terminal to the cleavage site is a different aliphatic amino acid. The clotting enzymes of all these species also act on a variety of synthetic chromogenic peptides in which the second and third amino acid positions can differ in limited ways from the native sequences.

In one embodiment of the present invention, as applied to the horseshoe-crab amebocyte lysate system, an intact substrate containing the native sequence from the C-terminus of T. tridentatus peptide C is used together with existing monoclonal antibodies that bind to this sequence.

In another embodiment of the present invention, as also applied to the horseshoe-crab amebocyte lysate system, an intact substrate containing the cleavage site-defining peptide linked to a hapten is used together with new monoclonal antibodies that bind to the hapten.

The first embodiment is specific to the Limulus amebocyte lysate system, while the second embodiment can be applied to a wide range of proteolytic cleavage sites in other systems merely by changing the site-defining amino acid sequence of the peptide-hapten substrate.

Embodiment I: Intact Substrate Containing a Native Sequence From T. tridentatus Peptide C

This embodiment allows for the fact that monoclonal antibodies against the C-terminal sequence of peptide C are more sequence-specific than the clotting enzymes from the four horseshoe crab species. Thus, an intact substrate with a sequence from Tachypleus or Carcinoscorpius spp. will bind its corresponding C-terminally reactive monoclonal antibody when exposed, while the monoclonal antibody will not be affected by the endogenous Limulus peptide C generated by the Limulus amebocyte lysate system. This consideration is relevant to analytical systems in which the horseshoe-crab amebocyte lysate and monoclonal antibody or other specific binding molecule are present at the same time, as may occur in the “latex” microparticle analytical format described below. They do not apply to systems in which the amebocyte lysate has been washed away before the specific binding molecule is applied, as in the ELISA format described below, nor do they apply to the use of amebocyte lysate from which the natural peptide-C-generating substrate, coagulogen, has been removed. In these cases it is of no significance whether the binding molecule could also bind to the native peptide.

Peptide synthesis: A series of peptides including up to ten C-terminal amino acid residues of peptide C from T. tridentatus were synthesized, with and without an N-terminal 4-azidobenzoyl group attached to permit photocoupling to a carrier. Synthesis was by the standard Fmoc-polyamide procedure in an automated peptide synthesizer. The synthesized peptides were:

| | |
|---|---|
| VAQESGVSGR | (SEQ ID NO:1) |
| AQESGVSGR | (SEQ ID NO:2) |
| QESGVSGR | (SEQ ID NO:3) |
| ESGVSGR | (SEQ ID NO:4) |
| SGVSGR | (SEQ ID NO:5) |
| GVSGR | (SEQ ID NO:6) |
| VSGR | (SEQ ID NO:7) |

plus their N-terminally substituted 4-azidobenzoyl analogs.

Conjugates for immunization and solid-phase coating: The octapeptide QESGVSGR (SEQ ID NO:3) was coupled with glutaraldehyde to two carrier proteins, purified protein derivative of tuberculin (PPD) and ovalbumin (OA), in molar ratios of peptide:carrier 1:1, 4:1; 5:1, 10:1 and 50:1. As the only available amino group on the peptide is the N-terminal amino group, coupling with glutaraldehyde will chiefly take place via this group, leaving the C-terminus of the peptide free.

4-azidobenzoyl-QESGVSGR (SEQ ID NO:3) was photocoupled to the same carriers in the same molar ratios. This also secures coupling via the N-terminus of the peptide, leaving the C-terminus free.

Immunization of mice: Separate groups of BCG-vaccinated mice were immunized with six doses (10 μg carrier) of each of these conjugates at 14-day intervals and their sera tested by ELISA in wells coated with the corresponding peptide coupled at molar ratios of 5:1 or 2.5:1 to the opposite carrier at a coating concentration of 10 μg/ml (with respect to carrier) in 0.05 M sodium carbonate buffer, pH 9.6.

Mice immunized with OA conjugates gave poor peptide-specific antibody responses. Mice immunized with PPD conjugates gave optimal peptide-specific antibody responses after the 3rd dose of 50:1 glutaraldehyde conjugate and after the 6th dose of 10:1 photocoupled conjugate.

Reactivity of antisera with peptides of different length: Determination of the cross-reactivity of the antisera with the peptides of different length by inhibition ELISA showed that the nonapeptide generally had >100% cross-reactivity. This implies that the octapeptide with a blocked N-terminus displays optimal reactivity with most of the antibodies. The pentapeptide and shorter peptides showed little or no cross-reactivity.

Monoclonal antibodies: Monoclonal antibodies against the octapeptide were prepared by standard methods from mice showing high humoral responses. This resulted in four monoclonal antibodies coded respectively 1, 2, 3 and 4. These showed the same pattern of cross-reactivity with peptides of different length as the antisera.

All the monoclonal antibodies reacted with endotoxin-activated T. tridentatus amebocyte lysate (TAL), antibody 2 showing the highest reactivity. Cross-reactivity with unactivated TAL was <0.1% and was estimated to be <0.025%. Cross-reactivity of the antibodies with endotoxin-activated Limulus amebocyte lysate (LAL) is <1% in comparison with activated TAL.

Synthesis of intact substrate peptides: The following intact substrate peptides are synthesized:

| | |
|---|---|
| QESGVSGRG | (SEQ ID NO:8) |
| QESGVSGRGF | (SEQ ID NO:9) |

Selection of monoclonal antibodies for reactivity with the free C-terminus of the QESGVSGR (SEQ ID NO:3) octapeptide: The monoclonal antibodies against the octapeptide QESGVSGR (SEQ ID NO:3) are checked for reactivity with N-terminal biocytin conjugated peptides SEQ ID NO:3, 8 and 9 bound to streptavidin-coated microwells. This identifies the antibodies that do not react with the C-terminally extended intact substrate peptides, i.e. those that react specifically with the free C-terminus of the cleaved substrate octapeptide. These are the antibodies subsequently used in the assay.

Embodiment II: Intact Peptide-Hapten Substrate with an Artificial Hapten Preceded by the Site-Defining Amino-Acid Sequence

Using this method, an analytical system is created that can be applied to a wide variety of different site-specific proteolyses by using the same binding molecule, which binds to a newly exposed invariable hapten to which different site-defining specific sequences can be linked by means of a peptide or ester bond that is split by the specific proteolytic enzyme.

Peptide synthesis: Artificial intact peptide substrates for the horseshoe-crab amebocyte lysate system, whether from Atlantic or Pacific horseshoe-crab species, are synthesized with the general sequence N-t-BOC-or N-Ac-[Val/Ile/Leu]-[Ser/Leu]-Gly-Arg-[hapten]-biocytin. The sequence N-terminal to the hapten is the N-terminally blocked amino-acid sequence that defines the specific cleavage site at the C-terminus of peptide C. The hapten may include or consist of a characteristic chemical moiety that does not occur in living tissues in order to promote the formation of highly specific binding molecules of high affinity for the hapten. If the hapten is a peptide, it will typically include a non-naturally occurring chemical moiety plus at least 3 and more preferably at least 7 amino acid residues. The unextended hapten is also synthesized and conjugated to a carrier via its C-terminus or other site remote from the amino group through which it is to be linked to the site-defining sequence in order to generate monoclonal antibodies against its free form by established methods.

Selection of monoclonal antibodies: Monoclonal antibodies to the unextended hapten are selected for specific binding of high affinity to the free hapten and for absence of binding to the intact substrate, in which the N-terminus or corresponding amino group of the hapten is blocked by the site-defining amino acid sequence.

ELISA Format for Horseshoe-Crab Amebocyte Lysate Using Substrate of Embodiment I or Embodiment II

The intact substrate is bound via its biocytinylated terminus to a streptavidin-coated solid phase. This exposes the opposite end to specific cleavage by clotting enzyme in endotoxin-activated LAL. The generation of the free terminus of the cleaved substrate thus exposed is then detected by specific binding of the selected monoclonal antibody.

All apparatus and solutions are rendered pyrogen-(endotoxin-) free by standard methods well known to those skilled in the art.

Polystyrene microwells are coated with streptavidin and the chosen biocytinylated substrate peptide is applied as the second layer. The wells are emptied and washed. Aqueous dilutions of endotoxin standards and unknowns (50 μl/well) are placed in the wells and the appropriate dilution of LAL reagent in LAL buffer is added (50 μl/well). The mixtures are incubated for a determined time at room temperature or at 37° C., whereupon the wells are emptied and washed. A dilution of the chosen monoclonal antibody labeled with horseradish peroxidase is added (100 μl/well), incubated for a determined time at room temperature, and the wells are emptied and washed. A chromogenic substrate solution for peroxidase, containing 3,3′,5,5′-tetramethylbenzidine or other appropriate chromogenic reagent, is added (100 μl/well), incubated for a determined time, and the color reaction stopped by adding with 0.5 M sulfuric acid (100 μl/well). The optical densities of the solutions in the wells are read in an ELISA reader at an appropriate wavelength for the colored product.

Within a low concentration range the optical densities obtained will be proportional to LAL activation and hence to the endotoxin activity present in the sample. This is quantified by comparison with the optical densities obtained with dilutions of control standard endotoxin.

Latex Microparticle Assay Format for Horseshoe-Crab Amebocyte Lysate Using Substrate of Embodiment I or Embodiment II

In this assay format a first population of latex (polystyrene) microspheres or microparticles in suspension is coated with streptavidin followed by the biocytinylated intact substrate, while a second population of microspheres or microparticles is coated with a monoclonal antibody that reacts with the newly exposed free terminus of the cleaved substrate. Addition of sample and a dilution of LAL to a mixture of the microsphere suspensions generates antibody-binding free termini on the substrate-coated microspheres, depending on the degree of LAL activation by endotoxin. This brings about an agglutination of these particles with the antibody-coated particles, giving rise to a signal that can be detected by a change in the light scattering properties of the suspension. Quantification is by comparison with results obtained from dilutions of control standard endotoxin.

This method can readily be adapted to automation in a flow system, allowing for rapid analysis of individual samples.

It is to be expected that the sensitivities of the above assays will be further improved by using LAL from which the native substrate, LAL coagulogen, has been removed.

The assays can be rendered specific for endotoxin by prior treatment of the LAL with an excess of laminarin. The assays can also be rendered specific for β-D-glucans by removal of factor C by means of a monoclonal antibody against that factor.

The following nonlimiting examples are provided to further illustrate experiments performed relating to the present invention.

### EXAMPLES

In the following experiments the following synthetic peptides have been used:

| | |
|---|---|
| KVIVSQETKDKIE | (SEQ ID NO:10) |
| Biocytin-VQAITDKDEISGR | (SEQ ID NO:11) |
| Biotin-VQAITDKDEISGRGF | (SEQ ID NO:12) |
| KVQAITDKDEISGRGF | (SEQ ID NO:13) |
| DKDEISGR | (SEQ ID NO:14) |
| ITDKDEISGR | (SEQ ID NO:15) |
| Biocytin-VQAITDKDEISGRGF | (SEQ ID NO:16) |

### Example 1

### Testing for Antibody Specificity

MaxiSorp 96-well microtiter plates (Nunc) were coated for two hours at 20-25° C. with 100 μl/well of 0.25 μg/ml streptavidin (Zymed). The plates were then washed for 4×2 minutes with wash buffer (phosphate-buffered saline (PBS), pH 7.4, containing 0.05% v/v Tween 20). The peptides (SEQ ID NO:10 & 11) were made up to a concentration of 1.0 μg/ml in PBS and 100 μl of the respective solutions were added to each well of the appropriate rows. The plates were incubated at 20-25° C. for one hour and then washed as before. Dilution buffer (wash buffer containing 0.5% w/v bovine albumin) was added to each well at 100 μl per well. Culture supernatant containing monoclonal antibody HYB 100-01 (HYB 100-01 CS) was diluted one part in 50 of dilution buffer and 100 μl added to well 1 of the appropriate rows. Serial doubling dilutions (100 μl in 200 μl) were then carried out across the plate leaving the last well in each row as a blank without antibody. The plates were incubated at 20-25° C. for one hour and then washed as previously. Peroxidase-conjugated rabbit anti-mouse immunoglobulin antibody (Dako) diluted 1 in 2000 in dilution buffer was added to each well at 100 μl per well. The plates were incubated at 20-25° C. for one hour and then washed as previously. The plates were developed by the addition to each well of 100 μl of ortho-phenylenediamine (OPD) substrate solution containing OPD at 0.4 mg/ml and 35% hydrogen peroxide 0.4 μl/ml in 65 mM phosphate/35 mM citrate buffer, pH 5.0, followed by incubation in the dark for 10-15 minutes. The color reaction was stopped by adding 100 μl of 1 M sulfuric acid to each well and the absorbance measured at 492 nm in a microplate reader, subtracting the absorbance at 620 nm.

### Example 2

### Blocking HYB 100-01 Binding by Extending the C-Terminus of Peptide SEQ ID NO:11 with Gly-Phe (Peptide SEQ ID NO:12)

A MaxiSorp 96-well microtiter plate (Nunc) was coated at 4° C. with 100 μl/well of 0.25 μg/ml streptavidin (Zymed) in 0.05 M sodium carbonate buffer. pH 9.6. The plate was then washed 4×2 minutes with wash buffer. PBS (100 μl) was added to each well of the plate. The peptides SEQ ID NO:11 & 12 were made up to a concentration of 100 μg/ml in PBS and 25 μl of the respective solutions were added to rows A and E of the plate. Serial one-in-five dilutions (25 μl in 125 μl) were then carried out down the next two rows (B and C, and F and G, respectively. The plate was incubated at 20-25° C. for one hour and washed as before. Dilution buffer (100 μl) was added to each well of the plate. Culture supernatant containing the monoclonal antibody HYB 100-01 (HYB 100-01 CS) was diluted one part in 5 of dilution buffer and 100 μl added to all wells of column 1, whereupon serial dilutions (100 μl in 200 μl) were carried out across the plate, leaving the last well in each row as a blank without antibody. The plate was then incubated at 20-25° C. for one hour and washed as before. Peroxidase-conjugated rabbit anti-mouse immunoglobulin antibody (Dako) diluted 1 in 2000 in dilution buffer was added to each well at 100 μl per well. The plates were incubated at 20-25° C. for one hour and then washed as previously. The plates were developed by the addition to each well of 100 μl of TMB ONE substrate solution (Kem-En-Tec) containing 3,3′,5,5′-tetramethylbenzamidine, followed by incubation in the dark for 10-15 minutes. The color reaction was stopped by adding 100 μl of 1 M sulfuric acid to each well and the absorbance measured at 450 nm in a microplate reader, subtracting the absorbance at 620 nm.

### Example 3

### Examination of the Effects of Peroxidase Conjugation on the Signal Given by HYB 100-01

A MaxiSorp 96-well microtiter plate (Nunc) was coated at 4° C. with 100 μl/well of 0.25 μg/ml streptavidin (Zymed) in 0.05 M sodium carbonate buffer. pH 9.6. The plate was then washed 4×2 minutes with wash buffer. PBS (100 μl) was added to each well of the plate. Peptide SEQ ID NO:11 was made up to a concentration of 100 ng/ml in PBS and 100 μl were added to each well. The plate was incubated at 20-25° C. for one hour and then washed as before. Dilution buffer (100 μl) was added to each well. Protein-G affinity-purified HYB 100-01 (HYB 100-01) and peroxidase-conjugated HYB 100-01 (HYB 100-01-POD) (Biotrend) were diluted to a concentration of 4 μg/ml in dilution buffer. Either the HYB 100-01 or the HYB 100-01-POD solution was added to relevant wells of column 1 at 100 μl per well. Serial dilutions (100 μl in 200 μl) were carried out across the plate leaving the last well in each row as a blank without antibody. The plate was then incubated at 20-25° C. for one hour and washed as before. To each well containing non-conjugated HYB 100-01 was added 100 μl of peroxidase-conjugated rabbit anti-mouse immunoglobulin antibody (Dako) diluted 1 in 2000 in dilution buffer, while dilution buffer alone (100 μl) was added to the other wells. The plate was incubated at 20-25° C. for one hour and then washed as before. The plate was developed by the addition to each well of 100 μl of TMB ONE substrate solution (Kem-En-Tec) containing 3,3′,5,5′-tetramethylbenzamidine, followed by incubation in the dark for 10-15 minutes. The color reaction was stopped by adding 100 μl of 1 M sulfuric acid to each well and the absorbance measured at 450 nm in a microplate reader, subtracting the absorbance at 620 nm.

### Example 4

### Testing for TAL Activation on Streptavidin-Coated Plates

Under precautions to keep endotoxin contamination to a minimum, a solution of peptide (SEQ ID NO:12) at a concentration of 40 ng/ml in LAL reagent water (LRW) (Cambrex) was added to all wells of a streptavidin Immobilizer 96-well microtiter plate (Nunc) at 100 μl per well. After incubation for 10 minutes at 20-25° C., 100 μl of Control Standard Endotoxin (CSE) solution of 10 EU/ml were added to the first well of the appropriate rows. Serial doubling dilutions were then carried out across the plate leaving the last well as a blank without endotoxin. A 0.5-ml vial of TAL reagent (Zhanjiang A&C Biologicals Ltd) was reconstituted in 0.5 ml of LRW. This was diluted 12-, 15-, 30- and 40-fold in endotoxin-free 50 mM Tris buffer (Cambrex), whereupon 100 μl of a given dilution were added to each well of the corresponding row. The microtiter plate was incubated at 37° C. for 45 minutes. The reaction was stopped by quickly emptying the wells followed by 3×2 minute washes with stop buffer (25% isopropanol v/v in 100 mM glycine-HCl, pH 2.4). The plate was then equilibrated with 2×10-30-second washes with wash buffer. HYB 100-01-POD was diluted to 0.5 μg/ml in dilution buffer and added to each well at 100 μl/well. The plate was then incubated at 20-25° C. for one hour and washed 3×2 minutes with wash buffer. The plate was developed by the addition to each well of 100 μl of TMB ONE substrate solution (Kem-En-Tec) containing 3,3′,5,5′-tetramethylbenzamidine, followed by incubation in the dark for 10-15 minutes. The color reaction was stopped by adding 100 μl of 1 M sulfuric acid to each well and the absorbance measured at 450 nm in a microplate reader, subtracting the absorbance at 620 nm.

### Example 5

### Testing the Ability of HYB 100-01-POD to Bind to Peptides Coupled Directly to the ELISA Plate

The appropriate rows of an Immobilizer 96-well microtiter plate (Nunc) were coated for 2 hours at 20-25° C. with 100 μl per well of either PBS (blank) or PBS containing 31.25 ng/ml peptide (SEQ ID NO:11). The plate was then washed 4×2 minutes with wash buffer. Dilution buffer (100 μl) was added to each well. Peroxidase-conjugated HYB 100-01 (HYB 100-01-POD) was diluted to 4 μg/ml in dilution buffer and added to relevant wells of column 1 at 100 μl per well. Serial doubling dilutions were then carried out across the plate, leaving the last well in each row as a blank without antibody conjugate. The plate was incubated at 20-25° C. for one hour and washed as previously. The plate was then developed by the addition to each well of 100 μl of TMB ONE substrate solution (Kem-En-Tec) containing 3,3′,5,5′-tetramethylbenzamidine, followed by incubation in the dark for 10-15 minutes. The color reaction was stopped by adding 100 μl of 1 M sulfuric acid to each well and the absorbance measured at 450 nm in a microplate reader, subtracting the absorbance at 620 nm.

### Example 6

### Examining the Effect of Peptide Length and N-Terminus on the Signal Obtained with TAL Reagent and HYB 100-01-POD

PBS (100 μl) was added to all wells of an Immobilizer 96-well microtiter plate (Nunc). The relevant peptides (SEQ ID NO:11, 13, 14, 15 and 16) were all diluted to 50 μg/ml in PBS and 100 μl of individual solutions added to the first well of the respective row. Serial doubling dilutions (100 μl in 200 μl) were then carried out across the plate leaving the last well in each row as a blank without peptide. The peptides were allowed to couple to the plate for two hours at 20-25° C. Residual coupling activity was reduced by the addition of 50 μl of 50 mM Tris Buffer, pH 8.0, for 30 minutes. Each well was then washed with 100 μl of PBS. To 0.5 ml of TAL reagent (Zhanjiang A&C Biologicals Ltd) was added 15 EU of Control Standard Endotoxin (CSE) followed by 12 ml of endotoxin-free PBS, and 100 μl of this solution was then added to each well. The plate was then incubated at 37° C. for 45 minutes. The reaction was stopped by rapidly emptying the wells followed by 3×2 minute washes with stop buffer (25% v/v isopropanol in 200 mM glycine-HCl₁, pH 2.6). The plate was then equilibrated with 2×10-30-second washes with wash buffer. HYB 100-01-POD was diluted to 0.5 μg/ml in dilution buffer and 100 μl added to each well. The plate was then incubated at 20-25° C. for 30 minutes and washed 3×2 minutes with wash buffer. The plate was developed by the addition to each well of 100 μl of TMB ONE substrate solution (Kem-En-Tec) containing 3,3′,5,5′-tetramethylbenzamidine, followed by incubation in the dark for 10 minutes. The color reaction was stopped by adding 100 μl of 1 M sulfuric acid to each well and the absorbance measured at 450 nm in a microplate reader, subtracting the absorbance at 620 nm.

### Example 7

### Endotoxin Measurement with TAL Reagent and Substrate Peptide Coupled Directly to the Microtiter Plate

Under precautions to keep endotoxin contamination to a minimum, a solution of peptide (SEQ ID NO:13) at a concentration of 0.3 μg/ml in PBS was added to all wells of an Immobilizer 96-well microtiter plate (Nunc) at 100 μl per well. The peptide was allowed to couple to the plate overnight at 4° C. Residual coupling activity was reduced by the addition to each well of 50 μl of endotoxin-free 50 mM Tris buffer (Cambrex) for 15 minutes. The plate was then emptied and 100 μl of the appropriate Control Standard Endotoxin (CSE) in LAL reagent water (LRW) (Cambrex) was added to the appropriate wells. A 0.5-ml vial of TAL reagent (Zhanjiang A&C Biologicals Ltd) was reconstituted in 6 ml of endotoxin-free 50 mM Tris buffer. This was then vortexed for exactly 30 seconds and 50 μl added to each well of the appropriate rows. The microtiter plate was incubated at 37° C. for 30 minutes. The reaction was stopped by quickly emptying the wells followed by 3×2 minute washes with stop buffer (25% v/v isopropanol in 200 mM glycine-HCl, pH 2.6). The plate was then equilibrated with 2×10-30-second washes with wash buffer. HYB 100-01-POD was diluted to 0.1 μg/ml in dilution buffer and 100 μl added to each well. The plate was then incubated at 20-25° C. for 30 minutes and washed 3×2 minutes with wash buffer. The plate was developed by the addition to each well of 100 μl of TMB ONE substrate solution (Kem-En-Tec) containing 3,3′,5,5′-tetramethylbenzamidine, followed by incubation in the dark for 10 minutes. The color reaction was stopped by adding 100 μl of 1 M sulfuric acid to each well and the absorbance measured at 450 nm in a microplate reader, subtracting the absorbance at 620 nm.

### Example 8

### Development of a TAL-Based Endotoxin ELISA

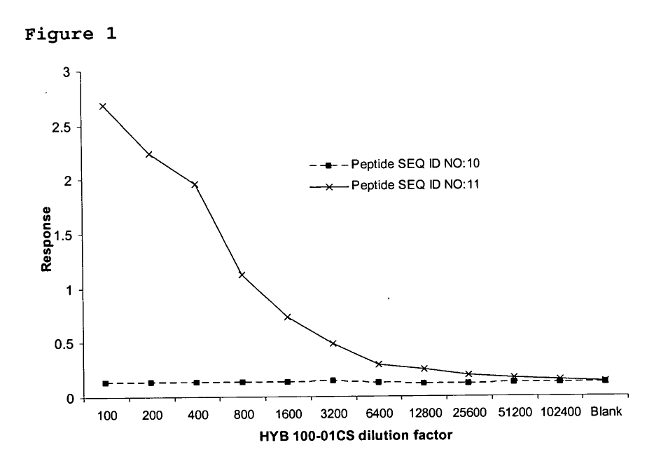

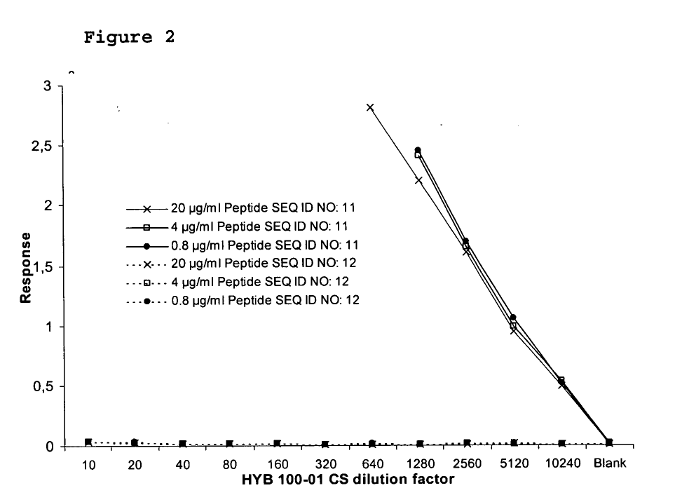

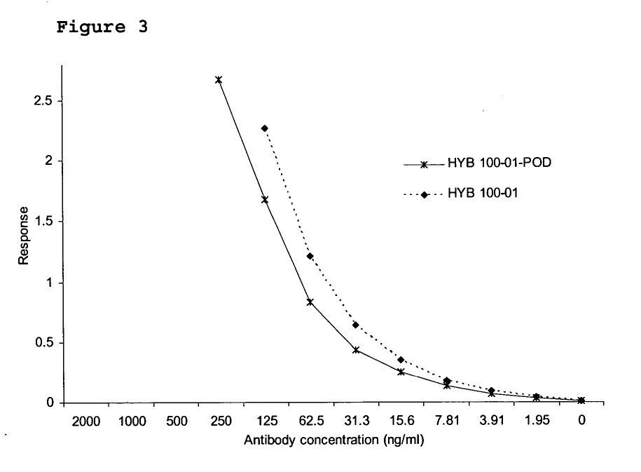

Monoclonal antibody HYB 100-01 was raised against a 27-amino-acid synthetic peptide equivalent to peptide c liberated from LAL coagulogen upon activation. HYB 100-01 was shown to bind specifically to activated LAL adsorbed to the surface of a microtiter well (Zhang G H, Baek L, Nielsen P E, Buchardt 0, Koch C, 1994, Sensitive quantitation of endotoxin by enzyme-linked immunosorbent assay with monoclonal antibodies against Limulus peptide C, Journal of Clinical Microbiology 32:416-22). It has been shown in Example 1 (see FIG. 1) that the monoclonal antibody HYB 100-01 binds an N-terminally biotinylated peptide (SEQ ID NO:11) representing the C-terminal region of LAL coagulogen-derived peptide c, which has been bound to a streptavidin-coated microtiter plate. HYB 100-01 did not bind a C-terminally biotinylated peptide (SEQ ID NO:10) representing the N-terminal region of LAL coagulogen-derived peptide c. However, it has been found in Example 2 (see FIG. 2) that although HYB 100-01 binds a peptide (SEQ ID NO:11) representing the C-terminal region of LAL coagulogen-derived peptide c, it shows no appreciable binding to a peptide (SEQ ID NO:12) in which the C-terminus of peptide (SEQ ID NO:11) has been extended by the next two amino-acid residues downstream from cleavage site at the C-terminus of peptide c in LAL coagulogen. For ease of use HYB 100-01 was conjugated with peroxidase. In Example 3 (see FIG. 3) the conjugate (HYB 100-01-POD) gives a signal, when bound to immobilized peptide SEQ ID NO:11, that is slightly lower but still comparable with that obtained with the unconjugated antibody developed with peroxidase-conjugated rabbit anti-mouse immunoglobulin antibody.

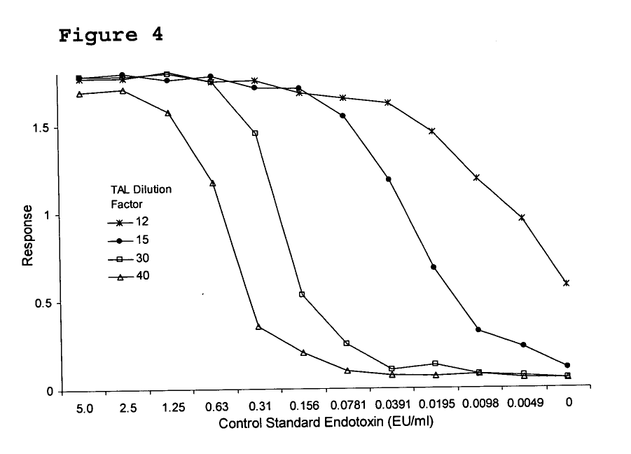

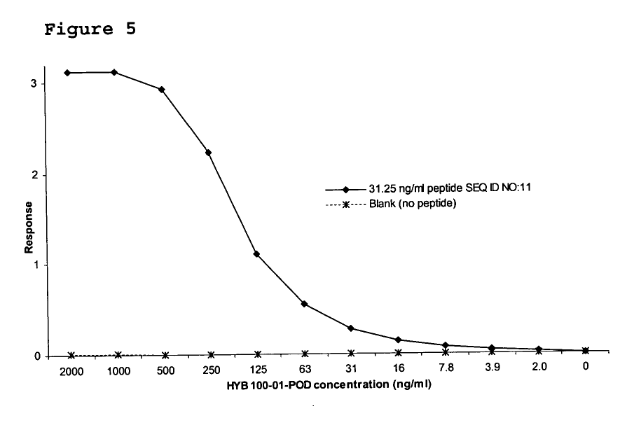

Monoclonal antibody HYB 100-01 is thus able to bind the free C-terminus of LAL coagulogen-derived peptide c and this binding is blocked by the addition of two extra amino-acid residues to the C-terminus. Site-specific proteolysis will cleave these two residues from the immobilized peptide and expose the free C-terminus to which HYB 100-01 or HYB 100-01-POD binds. To achieve this either LAL or TAL can be used. Example 4 (see FIG. 4) shows how the activation of TAL with endotoxin results in the site-specific cleavage of the immobilized substrate peptide (SEQ ID NO:12) so that the resulting peptide can be detected by the HYB 100-01-POD antibody conjugate. Example 4 shows the sensitivity achievable with this analytical methodology. A control endotoxin concentration of 0.005 EU/ml is detectable even when the standard TAL reagent is diluted by a factor of 15. The sensitivity is increased even further when the TAL reagent is only diluted by a factor of 12. However, at this sensitivity, contamination of the materials and reagents used with traces of endotoxin increases the background response. The analytical procedures have therefore been adjusted in the first instance to allow the reliable detection of endotoxin at a concentration of 0.005 EU/ml. In one procedure, the substrate peptide is biotinylated at its N-terminus and bound via the biotin moiety to streptavidin immobilized on the surface of the microtiter well. In another, preferred, procedure, the use of streptavidin, which is a probable source of endotoxin contamination, is avoided by coupling the substrate peptide directly to an Immobilizer microtiter plate (Nunc). Chemical groups present on the Immobilizer plate surface couple covalently to free amino groups present on the peptide. In Example 5 (see FIG. 5), free peptide (SEQ ID NO:11) was found to couple to the Immobilizer plate and could be detected with HYB 100-01-POD.

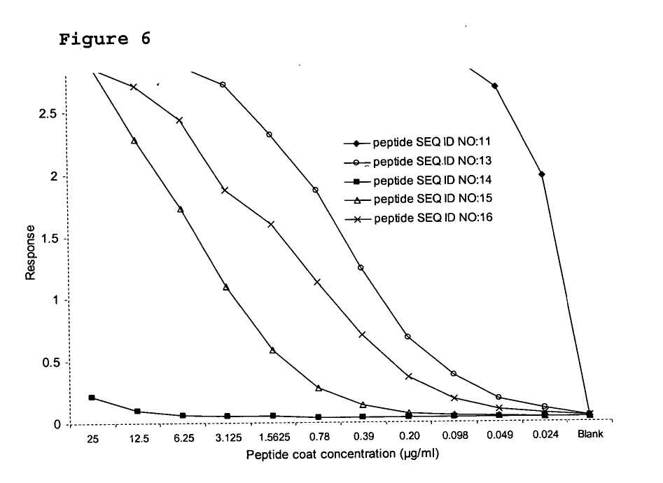

In Example 6 (see FIG. 6) the influence of the length of the immobilized peptide on the signal obtained with this analytical procedure was examined. Peptide SEQ ID NO:11 gave a much stronger signal than the shorter peptides (SEQ ID NO:14 & 15), a maximal signal being reached at a coating concentration of about 0.05 μg/ml. Peptide SEQ ID NO:15, lacking the biocytin and three N-terminal residues present in peptide SEQ ID NO:11, only gave a similar signal at a coating concentration of 12.5-25 μg/ml. If a further two amino-acid residues are removed from the N-terminus, as in peptide SEQ ID NO:14, the signal is almost totally lost. This indicates that peptide SEQ ID NO:11 cannot be shortened without diminishing the binding of monoclonal antibody HYB 100-01-POD. The effect of N-terminal biocytin on the response was also examined with the substrate peptide (SEQ ID NO:16) that lacks this moiety. This gave a slightly lower signal than peptide SEQ ID NO:13, which does contain the biotinylated epsilon amino group. As the two peptides are otherwise similar, the difference in response is proposed to be due to a slight lower coupling efficiency in the absence of biocytin. The lack of significant signal in the blank wells without peptide shows that the signal depends on the presence of the substrate peptide as no significant response is found in the blank (no peptide).

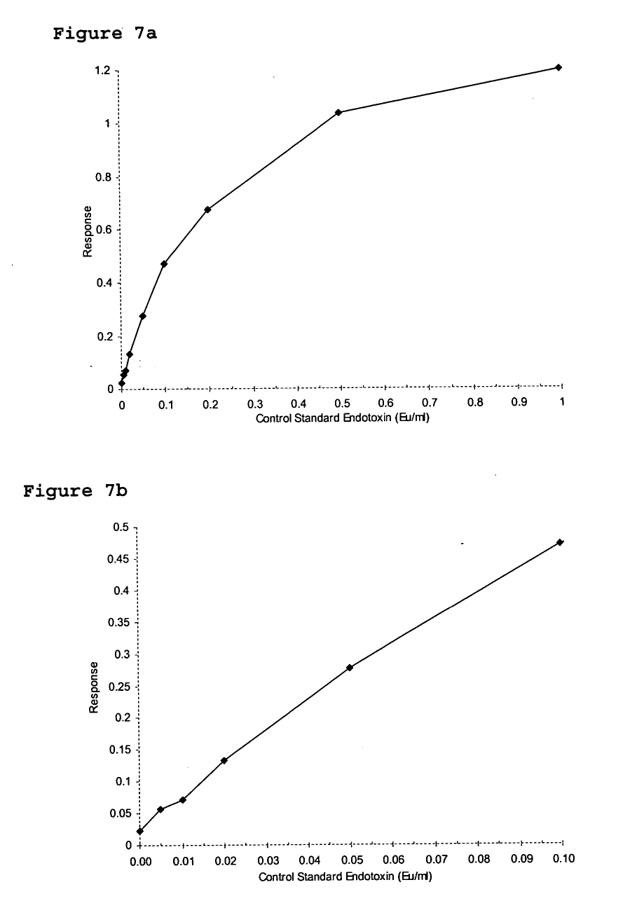

Example 7 (see FIGS. 7a and 7b) illustrates the use of the preferred procedure, in which the substrate peptide (SEQ ID NO: 13) is coated directly to the microtiter plate and not via streptavidin. In this system endotoxin concentrations from 0.005 EU/ml to 0.5 EU/ml can be measured with TAL reagent diluted 1 in 12 and incubated for only 30 minutes.

1. A method for determining activity or concentration of an analyte in a sample based upon site-specific enzymatic cleavage of an intact substrate by the analyte or by an enzyme activated by the presence of the analyte, comprising:
(a) generating or selecting a binding molecule specific for a free end of a newly exposed terminal sequence of the intact substrate following proteolytic cleavage of the intact substrate by the analyte or by an enzyme activated by the presence of the analyte;
(b) contacting a sample suspected of containing the analyte or an enzyme activated by the presence of the analyte with the intact substrate under conditions which promote or induce site-specific proteolytic cleavage of said intact substrate by the analyte or by an enzyme activated by the presence of the analyte, said intact substrate being attached to a continuous or particulate solid phase;
(c) contacting the site-specifically cleaved substrate with the binding molecule of step (a); and
(d) measuring binding of the binding molecule to the site-specifically cleaved substrate to determine activity or concentration of the analyte in the sample.

2. The method of claim 1 wherein the intact substrate comprises an amino acid sequence defining the site of specific proteolytic cleavage linked to a hapten by a bond susceptible to the specific proteolytic cleavage, said hapten being a chemical compound against which binding molecules of high specificity and affinity can be generated which do not react with the intact substrate.

3. The method of claim 1 wherein the binding molecule is linked to an amplification system to increase sensitivity.

4. The method of claim 1 wherein the intact substrate is linked to a continuous or particulate solid phase.

5. The method of claim 1 wherein the binding molecule is linked to a continuous or particulate solid phase.

6. The method of claim 1 wherein the intact substrate is coated onto a first population of microparticles or microspheres and the binding molecule is coated onto a second population of microparticles or microspheres and site-specific cleavage of the intact substrate is detected by measuring aggregation of particles from the first and second populations.

7. The method of any of claims 1 through 6 applied to the measurement of the endotoxin content in a sample with aid of horseshoe-crab amebocyte lysate.

8. The method of any of claims 1 through 6 applied to the measurement of site-specific protease activities in samples of bodily fluids.


SEQUENCE LISTING (16 sequences)

SEQ ID NO: 1 - protein, 10 aa, Artificial sequence
  Synthetic
VAQESGVSGR

SEQ ID NO: 2 - protein, 9 aa, Artificial sequence
  Synthetic
AQESGVSGR

SEQ ID NO: 3 - protein, 8 aa, Artificial sequence
  Synthetic
QESGVSGR

SEQ ID NO: 4 - protein, 7 aa, Artificial sequence
  Synthetic
ESGVSGR

SEQ ID NO: 5 - protein, 6 aa, Artificial sequence
  Synthetic
SGVSGR

SEQ ID NO: 6 - protein, 5 aa, Artificial sequence
  Synthetic
GVSGR

SEQ ID NO: 7 - protein, 4 aa, Artificial sequence
  Synthetic
VSGR

SEQ ID NO: 8 - protein, 9 aa, Artificial sequence
  Synthetic
QESGVSGRG

SEQ ID NO: 9 - protein, 10 aa, Artificial sequence
  Synthetic
QESGVSGRGF

SEQ ID NO: 10 - protein, 13 aa, Artificial sequence
  Synthetic
KVIVSQETKDKIE

SEQ ID NO: 11 - protein, 13 aa, Artificial sequence
  Synthetic
VQAITDKDEISGR

SEQ ID NO: 12 - protein, 15 aa, Artificial sequence
  Synthetic
VQAITDKDEISGRGF

SEQ ID NO: 13 - protein, 16 aa, Artificial sequence
  Synthetic
KVQAITDKDEISGRGF

SEQ ID NO: 14 - protein, 8 aa, Artificial sequence
  Synthetic
DKDEISGR

SEQ ID NO: 15 - protein, 10 aa, Artificial sequence
  Synthetic
ITDKDEISGR

SEQ ID NO: 16 - protein, 15 aa, Artificial sequence
  Synthetic
VQAITDKDEISGRGF